(19) United States
(12) Patent Application Publication
(10) Pub. No.: US 2018/0221126 A1
(43) Pub. Date: Aug. 9, 2018

(54) HERNIA REPAIR DEVICE, SYSTEM AND METHOD

(71) Applicant: MORENA MEDICAL LTD., Tel Aviv (IL)

(72) Inventor: Igor IGOV, Ramla (IL)

(21) Appl. No.: 15/749,814

(22) PCT Filed: Aug. 2, 2016
(86) PCT No.: PCT/IL2016/050846

§ 371 Date: Feb. 2, 2018

Related U.S. Application Data
(60) Provisional application No. 62/200,074, filed on Aug. 2, 2015.

(30) Foreign Application Priority Data
Jan. 25, 2016 (DE) 10 2016 201 022.0

(51) Int. Cl. A61F 2/00; A61B 17/068; A61B 17/122; A61B 17/128

(52) U.S. Cl. CPC A61F 2/0063; A61B 17/0682; A61B 2017/00477; A61B 17/1285; A61F 2002/0072; A61B 17/122

(57) ABSTRACT

A system comprising an elongated mesh deployment tool having a proximal tool part and a distal tool part. The system comprises one or more wires for coupling a surgical mesh with the distal tool part, where the wires are coupled to the surgical mesh and the distal tool part in one or more locations. The distal tool part is configured for deploying the surgical mesh at a hernia location, where the deploying is performed by pulling the one or more wires in a proximal direction to mount the surgical mesh in a spread configuration near the hernia location.

### CROSS REFERENCE TO RELATED APPLICATIONS

This application claims priority to U.S. Provisional Patent Application No. 62/200,074, filed Aug. 2, 2015, entitled “Mesh Deployment Device”, and to German Patent Application No. 102016201022.0 filed Jan. 25, 2016, entitled “Hernia Repair Device, System, and Method”, the contents of both are incorporated herein by reference in their entirety.

### FIELD OF THE INVENTION

The invention relates to the field of hernia repair.

### BACKGROUND

Hernia repair refers to a surgical operation for the correction of a hernia, such as a bulging of internal organs or tissues through the wall that contains it. Hernias may occur in many places, including the abdomen, groin, diaphragm, brain, and at the site of a previous operation. There are many different approaches to the surgical repair of hernias, including herniorrhaphy, hernioplasty, and herniotomy. Hernioplasty is often performed as an ambulatory procedure.

One differentiating factor in hernia repair is whether the surgery is done open, or laparoscopically. Open hernia repair is when an incision is made in the skin directly over the hernia. Laparoscopic hernia repair is when minimally invasive cameras and equipment are used and the hernia is repaired with only small incisions. Another differentiating factor is whether a mesh is employed or not for treating the hernia.

A hernioplasty may be performed with an autogenous material, such as a patient's own tissue, or with a heterogeneous material, such as prolene mesh.

Surgical mesh used in hernioplasty is a loosely woven sheet which is used as either as permanent or temporary support for organs and other tissues. The meshes are available in both inorganic and biological materials, and are used in a variety of hernia surgeries. Though hernia repair surgery is the most common application, they may also be used to treat other conditions as well, such as pelvic organ prolapse.

Permanent meshes remain in the body, whereas temporary meshes dissolve over time. For example, TIGR® Matrix mesh was fully dissolved after three years in a recent trial on sheep. Some meshes combine permanent and temporary meshes such as Vipro, a product combining re-absorbable vipryl, made from polyglycolic acid, and prolene, a non-reabsorbable polypropylene.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools, devices, and methods which are meant to be exemplary and illustrative, not limiting in scope.

There is provided, in accordance with an embodiment, a laparoscopic hernia mesh repair system for repairing of hernias in a surgical cavity by deploying a surgical mesh. The system comprises a mesh deployment tool of longitudinal extension and having a first, proximal tool part and a second, distal tool part. The distal tool part may selectively be set in an insertion configuration for allowing minimally invasively inserting the distal tool part into the surgical cavity, and in an expanded configuration for attaching the surgical mesh to the distal tool part in a spread configuration allowing deployment of the mesh at a hernia site. The system comprises wires for coupling the surgical mesh with the distal tool part, where the wires are coupled to the mesh and the distal tool part at locations so that pulling of the wires into a proximal direction while the distal tool part is in the expanded configuration causes the mesh to be spread and mounted in the spread configuration onto the distal tool part.

Optionally, the wires extend from outside the surgical cavity into the surgical cavity for allowing spreading the mesh which is located in the surgical cavity while the distal tool part is outside the surgical cavity.

Optionally, the expanded configuration the distal tool part has a polygon-shaped frame-like structure.

Optionally, the distal tool part comprises sections that are bendable relative to each other to form the polygon-shaped frame-like structure.

Optionally, the distal tool part comprises two or more legs, where in the expanded configuration at least two of legs are pointing in angular directions which deviate from the longitudinal extension of the deployment tool and further different from each other so that the legs terminate in at least three end points for spreading a mesh.

Optionally, the legs comprise three or more legs that terminate in the expanded configuration in end points that lie in the same plane for spreading a mesh.

Optionally, the mesh is attached to the expanded distal part using, sutures, magnets, and/or suction.

Optionally, the laparoscopic hernia mesh repair system comprises anchors deployable by the distal tool part at the boundaries of the herniated tissue. When in the surgical cavity, the wires run from the surgical mesh to the anchors, and from the anchors to the distal tool section so that by pulling the wires, the mesh suspends from the anchors and is lifted towards the anchors for attachment at the herniated tissue.

Optionally, the laparoscopic hernia mesh repair system comprises a pulling mechanism for pulling the wires from outside the surgical cavity in a distal direction.

There is provided, in accordance with an embodiment, a laparoscopic hernia mesh repair system for repairing of hernias in a surgical cavity by deploying a surgical mesh. The system comprises a mesh deployment tool of longitudinal extension and having a first, proximal tool part and a second, distal tool part. The distal tool part may selectively be set in an insertion configuration for allowing minimally invasively inserting the distal tool part into the surgical cavity, and in an expanded configuration for attaching the surgical mesh to the distal tool part in a spread configuration allowing deployment of the mesh at a hernia site.

There is provided, in accordance with an embodiment, a system comprising an elongated mesh deployment tool having a proximal tool part and a distal tool part. The system comprises one or more wires for coupling a surgical mesh with the distal tool part, where the wires are coupled to the surgical mesh and the distal tool part in one or more locations. The distal tool part is configured for deploying the surgical mesh at a hernia location, where the deploying is performed by pulling the one or more wires in a proximal direction to mount the surgical mesh in a spread configuration near the hernia location.

Optionally, the one or more wires extends from outside a surgical cavity into the surgical cavity and are configured to spread the surgical mesh which is located in the surgical cavity while the distal tool part is outside the surgical cavity.

Optionally, expanded configuration the distal tool part has a polygon-shaped frame-like structure.

Optionally, the distal tool part comprises sections that are bendable relative to each other to form the polygon-shaped frame-like structure.

Optionally, the distal tool part comprises a plurality of legs, where in the expanded configuration at least two of the plurality of legs are oriented for spreading the surgical mesh.

Optionally, the plurality of legs comprises three or more legs that terminate in the expanded configuration in end points that lie substantially co-planar for spreading the surgical mesh.

Optionally, the mesh is attached to the expanded distal tool part using sutures, magnets, or suction.

Optionally, the system further comprises anchors deployable by the distal tool part at the boundaries of a herniated tissue, where in a surgical cavity the one or more wires runs from the surgical mesh to the anchors, and from the anchors to the distal tool section so that by pulling the one or more wires, the mesh suspends from the anchors and is lifted towards the anchors for attachment at the hernia location.

Optionally, the system further comprises a pulling mechanism located at the proximal end of the mesh deployment tool, where the pulling mechanism is configured for pulling the one or more wires from outside the surgical cavity in a proximal direction.

There is provided, in accordance with an embodiment, a system comprising an elongated mesh deployment tool having a proximal tool part and a distal tool part, where the distal tool part comprises a mesh clamp. The system comprises one or more wires for coupling a surgical mesh with the distal tool part, where the one or more wires are coupled to the surgical mesh and the distal tool part in one or more locations. The mesh clamp comprises (i) a collapsed configuration configured for minimally invasively insertion through a port into a surgical cavity, where the mesh clamp in the collapsed configuration grips a surgical mesh, and (ii) an expanded configuration configured for attaching the surgical mesh to the distal tool part, where the attaching is performed by pulling the one or more wires in a proximal direction to mount the surgical mesh in a spread configuration onto the distal tool part, and the expanded configuration is further configured for deploying the surgical mesh at a hernia location.

Optionally, the one or more wires extends from outside a surgical cavity into the surgical cavity and are configured to spread the surgical mesh which is located in the surgical cavity while the distal tool part is outside the surgical cavity.

Optionally, the system further comprises anchors deployable by the distal tool part at the boundaries of a herniated tissue, where in a surgical cavity the one or more wires runs from the surgical mesh to the anchors, and from the anchors to the distal tool section so that by pulling the one or more wires, the mesh suspends from the anchors and is lifted towards the anchors for attachment at the hernia location.

Optionally, the system further comprises a pulling mechanism located at the proximal end of the mesh deployment tool, where the pulling mechanism is configured for pulling the one or more wires from outside the surgical cavity in a proximal direction.

There is provided, in accordance with an embodiment, a system comprising an elongated mesh deployment tool having a proximal tool part and a distal tool part. The system comprises a plurality of legs, each leg extending from the distal tool part and configured to be strongly attracted to a magnet. Each leg comprises a slicing blade embedded therein, and each leg couples magnetically to a surgical mesh comprising a plurality of corresponding magnets secured to the surgical mesh, e.g., with thread. The plurality of legs comprises (i) a collapsed configuration configured for minimally invasively insertion through a port into a surgical cavity, and (ii) an expanded configuration configured for attaching the surgical mesh to the distal tool part, where the attaching is performed by mount the surgical mesh in a spread configuration onto the plurality of legs using magnetic attraction, and the expanded configuration is further configured for deploying the surgical mesh at a hernia location, and where when the surgical mesh is located at the hernia location the slicing blades slice each of the threads securing magnets to the surgical mesh.

Optionally, in the expanded configuration the distal tool part has a polygon-shaped frame-like structure.

Optionally, the distal tool part comprises sections that are bendable relative to each other to form the polygon-shaped frame-like structure.

There is provided, in accordance with an embodiment, a system comprising an elongated mesh deployment tool having a proximal tool part and a distal tool part, where the distal tool part comprises a plurality of bending sections and a plurality of suction orifices. Each suction orifice is configured to couple with part of a surgical mesh by applying suction to the proximal tool part. The distal tool part comprises (i) a collapsed configuration configured for minimally invasively insertion through a port into a surgical cavity, and (ii) an expanded configuration configured for attaching the surgical mesh in a spread configuration to the distal tool part by applying the suction, and the expanded configuration is further configured for deploying the surgical mesh at a hernia location.

Optionally, in the expanded configuration the distal tool part has a polygon-shaped frame-like structure.

Optionally, the distal tool part comprises sections that are bendable relative to each other to form the polygon-shaped frame-like structure.

There is provided, in accordance with an embodiment, a system comprising an elongated mesh deployment tool having a proximal tool part and a distal tool part, where the distal tool part comprises a plurality of bending sections. Each bending section is configured for attaching a surgical mesh using thread. The distal tool part comprises (i) a collapsed configuration configured for minimally invasively insertion through a port into a surgical cavity with the surgical mesh attached, and (ii) an expanded configuration configured for deploying the surgical mesh at a hernia location.

Optionally, in the expanded configuration the distal tool part has a polygon-shaped frame-like structure.

Optionally, the distal tool part comprises sections that are bendable relative to each other to form the polygon-shaped frame-like structure.

There is provided, in accordance with an embodiment, a system comprising a mesh deployment tool, where the mesh deployment tool comprises a proximal tool part and a distal tool part, where the distal tool part is configured to be inserted into a surgical cavity through a port, and where the distal tool part is configured to be expanded for attaching a surgical mesh to the distal tool part in a spread configuration allowing deployment of the surgical mesh at a hernia location.

Optionally, the system comprises a tacking mechanism located at the distal tool part configured for inserting tacks through the mesh into the tissue surrounding the hernia location.

Optionally, the tacks are configured to be biodegradable.

Optionally, the tacks are configured to be removed easily for repositioning the mesh.

Optionally, the tacks comprise a plurality of barbs, each barb of a different length.

In addition to the exemplary aspects and embodiments described above, further aspects and embodiments will become apparent by reference to the figures and by study of the following detailed description.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below:

FIG. 1 schematically shows a surgical tool for deployment of a surgical mesh;

FIG. 2 schematically shows a partially rigid tool with a flexible end for deployment of a surgical mesh;

FIG. 3 schematically shows a tool for inserting and deploying a surgical mesh with three flexible sections and three rigid sections;

FIG. 4 schematically shows a tool for inserting and deploying a surgical mesh with flexible and rigid sections in expanded configuration;

FIG. 5 schematically shows the tool of FIG. 4 in the collapsed configuration with the surgical mesh partially rolled around the tool;

FIG. 6 schematically shows a tool with four legs extending from the end of the tool;

FIG. 7 schematically shows surgical mesh and wires partially rolled or folded with the wires/sutures;

FIG. 8 schematically shows a second tool with a hook used to pull wires;

FIG. 9 schematically shows a clamp at the distal end of a surgical tool;

FIG. 10 schematically shows a surgical mesh rolled and clamped onto a tool;

FIG. 11 schematically shows a surgical mesh partially deployed inside a surgical cavity;

FIG. 12 schematically shows a second tool used to align the surgical mesh with a hernia location;

FIG. 13 schematically shows a surgical tool with two leg extensions for deploying a surgical mesh using wires/sutures;

FIG. 14 schematically shows surgical mesh attached to the hernia location with tacks;

FIG. 15 schematically shows a deployed surgical mesh being pulled by threads;

FIG. 16 schematically shows a surgical mesh being attached to a surgical tool using thread;

FIG. 17 schematically shows a front side of one leg of a multiple leg surgical tool;

FIG. 18 schematically shows a front and back side of one leg of a multiple leg surgical tool;

FIG. 19 schematically shows proximal and distal ends of a multiple leg surgical tool with a surgical mesh;

FIG. 20 schematically shows a surgical mesh being attached to a multiple leg surgical tool with magnets;

FIG. 21 schematically shows magnets that are attached to a jig with a mesh;

FIG. 22 schematically shows a close-up view of a leg of a surgical tool for clamping surgical mesh with magnets;

FIG. 23 schematically shows a cutting blade insert in the leg of a surgical tool;

FIG. 24 schematically shows a surgical tool with surgical mesh attached and anchors/tacks;

FIG. 25 is a schematic enlarged view of an example coupling element embodied by an anchor/tack;

FIG. 26 schematically shows a surgical tool with the surgical mesh attached by suction;

FIG. 27 schematically shows a collapsed configuration of a surgical tool for attaching the surgical mesh by suction;

FIG. 28 schematically shows an expanded configuration of a closed loop frame;

FIG. 29 schematically shows an expanded configuration of an open loop frame;

FIG. 30 schematically shows hooks in retracted position;

FIG. 31 schematically shows a hook in anchored position;

FIG. 32 schematically shows a hook deploying tool and a hook;

FIG. 33 schematically show rows of hooks connected with a thread;

FIG. 34 schematically shows a hook with a barbed point for entering a tissue;

FIG. 35 schematically shows a mesh coupling element delivery tool for deployment of a U-shaped coupling element;

FIG. 36A schematically shows a removable cross-shaped tack in a close configuration;

FIG. 36B schematically shows a removable cross-shaped tack in a partially open configuration;

FIG. 36C schematically shows a removable cross-shaped tack in an open configuration; and

FIG. 36D schematically shows a removable cross-shaped tack being removed.

FIG. 37 schematically shows a tool deploying a blocking element; and

FIG. 38 shows blocking element in collapsed configuration.

### DETAILED DESCRIPTION

According to some embodiments of the present invention, there are provided systems and methods for use in association with a hernia repair mesh. A surgical mesh is prepared for laparoscopic insertion, and inserted using a specially designed device for spreading or to span and securing the mesh to the herniation. The device may further prepare the mesh for tissue attachment by coupling, such as temporally, the mesh to herniated tissue prior to employing an attachment method and/or device permanently securing the mesh to the tissue. Such permanent mesh attachment may be obtained using for example suturing, stapling, clipping, gluing techniques, and/or the like.

Embodiments of the systems and methods provide the benefits of decreasing operation time, improved patient outcome from a better secured mesh, by reducing the openings required to be made when employing laparoscopic procedures, such as openings with 4 mm diameter or less, 3 mm or less, 2 mm or less, as well as comparably fewer openings, such as a maximum of 3 or 2 openings to be made into the abdominal wall, and/or the like. For example, the system disclosed herein allows delivering the mesh together with a mesh deployment tool, both in a closed or collapsed configuration into a body lumen or surgical cavity. In the surgical cavity, the deployment tool and the mesh are both expanded separately using the same or different tool. The mesh may be deployed and permanently attached at the hernia site. In another example, the mesh may be attached to the deployment tool in a collapsed, such as folded, rolled-up, and/or the like, configuration before entering the surgical cavity. The collapsible deployment tool may expand the mesh from the closed or collapsed configuration into an expanded, unrolled, unfolded, open, and/or the like configuration for placement of the mesh at the hernia site.

Exemplary embodiments are illustrated in referenced figures. Dimensions of components and features shown in the figures are generally chosen for convenience and clarity of presentation and are not necessarily shown to scale.

Optionally, a surgical mesh deployment system comprises an inserting tool, a deploying tool, a cutting tool, a grasping tool, an attachment tool, any combination thereof, and/or the like.

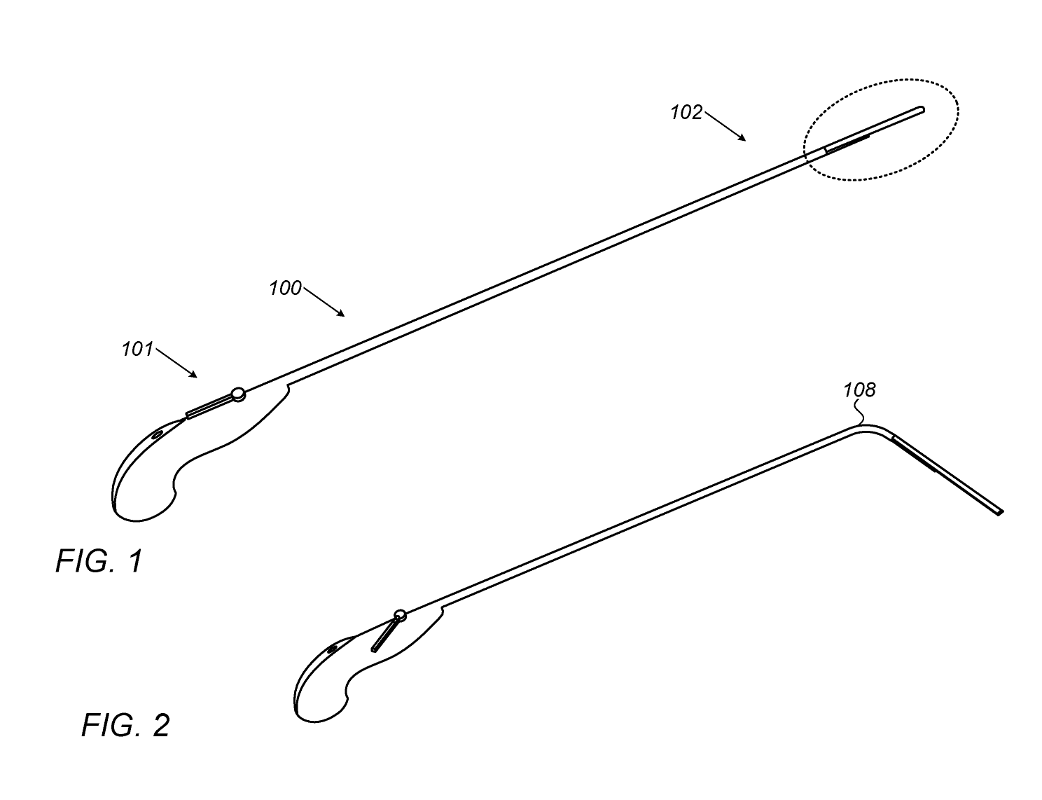

Optionally, a surgical mesh placement and deployment is performed by one or more rigid tools. For example, FIG. 1 schematically shows a surgical tool 100 for mesh deployment before insertion of a surgical mesh. A distal end 102 is configured to deploy a surgical mesh at a location of a hernia in a patient, while a proximal end is configured to be used by a surgeon to control the tool. The surgical mesh may be prepared in a rolled-up configuration before attaching to the distal end of the tool. The term “distal” refers to a location which is further away from a medical professional operating the system compared to a “proximal” location.

Optionally, a surgical mesh placement and deployment is performed by one or more partially rigid tools comprising a flexible section. For example, FIG. 2 schematically shows a partially rigid tool with a flexible end rotated 90 degrees at a flexible section 108. The end may be rotated relative to a viewing direction along the tool's longitudinal axis, from the proximal to the distal end of the tool, and in a direction which is normal to a plane in which a handle of the tool lies (i.e., from a top view of the tool).

Optionally, the deployment tool comprises a first and second tool part. The first tool part is used to roll up and couple the surgical mesh to the second tool part, and the second tool part is used to insert the mesh into the surgical cavity. For example, the second tool part is a clamp.

Optionally, a single legged device is used for deploying the surgical mesh at the site of herniation.

Optionally, the tool(s) are selectively set in a closed, collapsed, or insertion mode or configuration for insertion of a distal tool part into the surgical cavity, and an opened, expanded, or deployment mode or configuration for surgical mesh deployment.

Optionally, the mesh is attached to the tool(s), the tool is configured in a collapsed configuration, the tool(s) is laparoscopically inserted into surgical cavity and, in the surgical cavity, configured in an opened configuration. Optionally, the mesh is inserted in rolled-up configuration into the surgical cavity without using the deployment tool. For example, the mesh is inserted into the surgical cavity without being attached to the deployment tool. Once the mesh is in the surgical cavity, the deployment tool is inserted into the surgical cavity in collapsed configuration and only then deployed (e.g., expanded) for unrolling the rolled-up mesh. Optionally, the mesh may be coupled with wire(s) to the deployment tool before and after the mesh's deployment into the surgical cavity. For instance, the mesh may be delivered into the surgical cavity while the deployment is outside the cavity. However, wires may connect the mesh with the deployment tool. When the deployment tool is in the surgical cavity, manipulation (e.g., pulling) of the wires after the deployment tool is set in the expanded configuration may facilitate expanding and attaching the mesh to the expanded deployment tool.

For instance, the wires may pull the mesh towards the second tool part. For example, the mesh is attached with thread to the distal end of a tool in an expanded configuration, and then the tool is changed to a collapsed configuration for insertion into a surgical cavity with the mesh still attached. For example, through a port of a minimally invasive hernia repair procedure. Once inside, the tool may be converted to the expanded configuration, the mesh may be deployed at the hernia location, and the mesh may be detached from the tool.

Optionally, a surgical mesh placement and deployment is performed by one or more tools comprising a tool part which may be manipulated from a straight (or closed) configuration, in which the tool part extends along the longitudinal axis of the deployment tool, into another (open) configuration in which the tool part forms an expanded structure, such as a frame body or spread legs, that for example delineates in one plane a polygonal geometry, such as triangle, a hexagon, or the like. Optionally, the orientation of the plane in which the polygon lies in pivoted relative to the longitudinal axis.

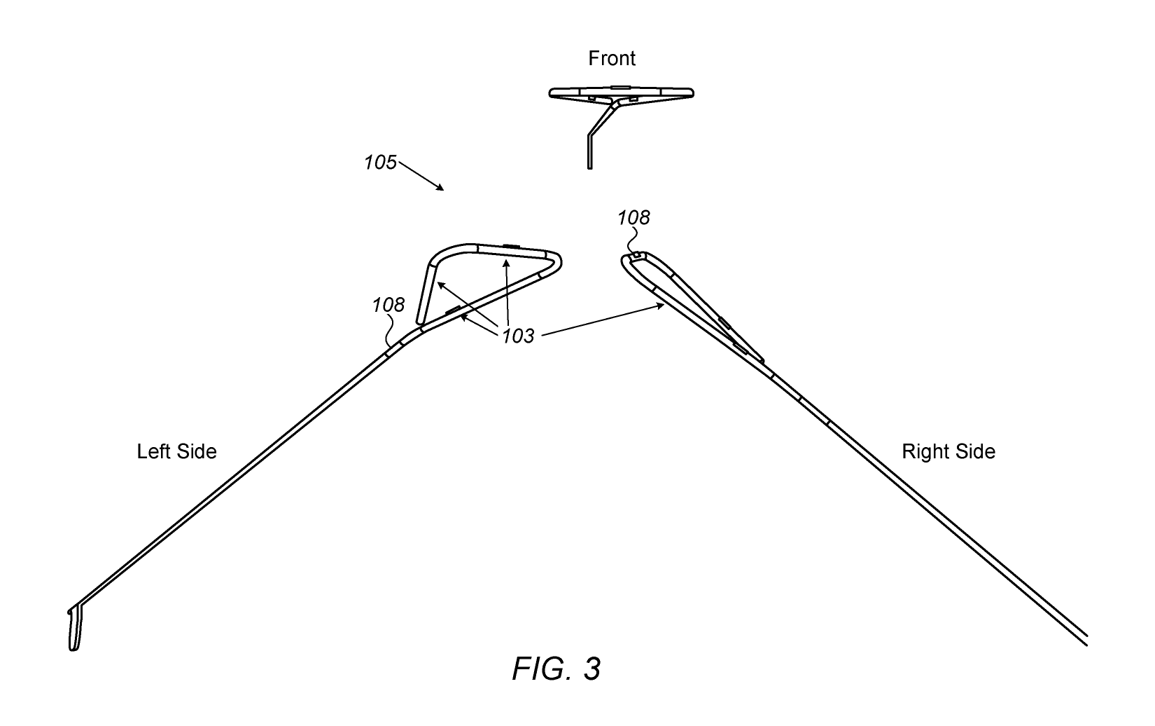

Optionally, the deployment tool comprises a tool part of which various sections may be bendable relative to each other to form the frame body. For example, the tool part may have two or more flexible sections with optionally rigid sections between them. For example, FIG. 3 schematically shows a tool 105 for inserting and deploying a surgical mesh with three flexible sections and three rigid sections. The three flexible sections 108 may be actuated so that the tool part forms a triangle-shaped frame structure from straight sections 103. Optionally, the surgical mesh is attached prior to surgery in the expanded configuration, the tool is then changed to the collapsed configuration, the tool is inserted into the surgical cavity, and the tool changed to the expanded configuration for expanding the mesh. Optionally, the surgical mesh is inserted into the surgical cavity while the deployment tool is outside the cavity. After the surgical mesh in placed in the surgical cavity, a part of the deployment tool is inserted into the cavity so that the tool part may be expanded into the frame structure. Optionally, when the tool part is set in the configuration of the frame structure, the mesh is pulled to the frame, expanded and attached to the frame.

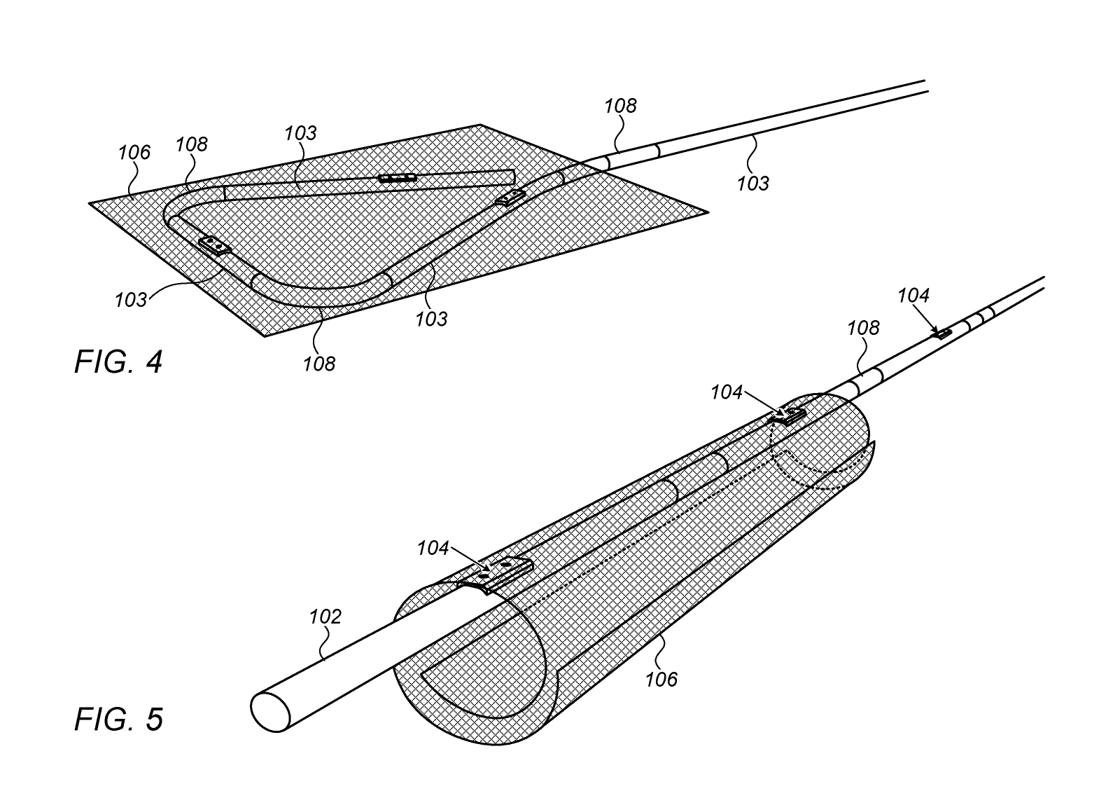

FIG. 4 schematically shows a tool for inserting and deploying a surgical mesh 106 with three flexible sections 108 and four rigid sections with a surgical mesh attached, where the tool is in the expanded configuration and the surgical mesh is spread in a substantially flat configuration. FIG. 5 schematically shows the tool of FIG. 4 in the collapsed configuration with the surgical mesh 106 partially rolled around the tool prior to insertion into a surgical cavity. Legs or clasps 104 are used to attach surgical mesh 106 to the tool.

Optionally, a surgical mesh placement and deployment is performed by tool(s) comprising a flexible end that is inserted in a collapsed configuration and is changed inside the surgical cavity to an expanded configuration.

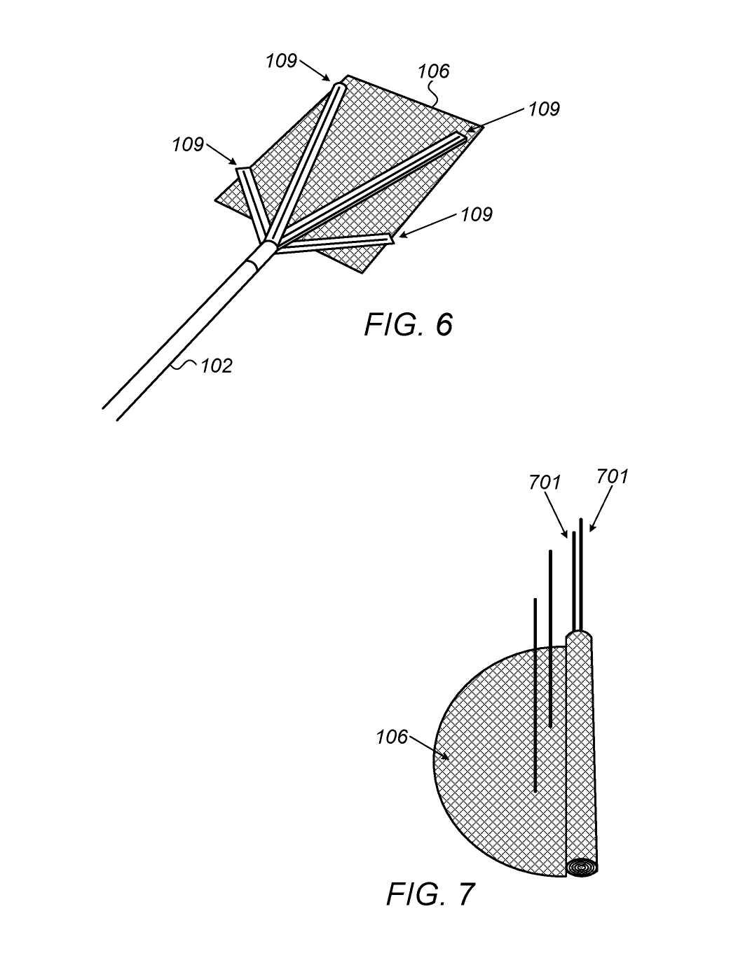

Optionally, a surgical tool end comprises multiple extensions for spreading a surgical mesh near a herniated tissue. For example, a multiple legged device is used for deploying the surgical mesh at the site of herniation. FIG. 6 schematically shows a tool with four legs 109 extending from the end of the tool. When the tool is changed to the expanded configuration inside the surgical cavity, the legs spread along with the surgical mesh 106.

Optionally, a placement and deployment are performed by a single tool.

Optionally, a placement is performed by a first tool and deployment is performed by a second tool.

Optionally, a surgical mesh is prepared with short wires, sutures, and/or the like, prior to insertion into a surgical cavity and the preparation assist in placing spreading and surgical mesh near the herniated tissue.

Optionally, a surgical mesh is prepared with long wires, sutures, and/or the like, prior to insertion into a surgical cavity and the preparation assist in placing and spreading the surgical mesh near the herniated tissue.

Optionally, staples, tacks, clips, hooks, and/or the like are placed using the tool.

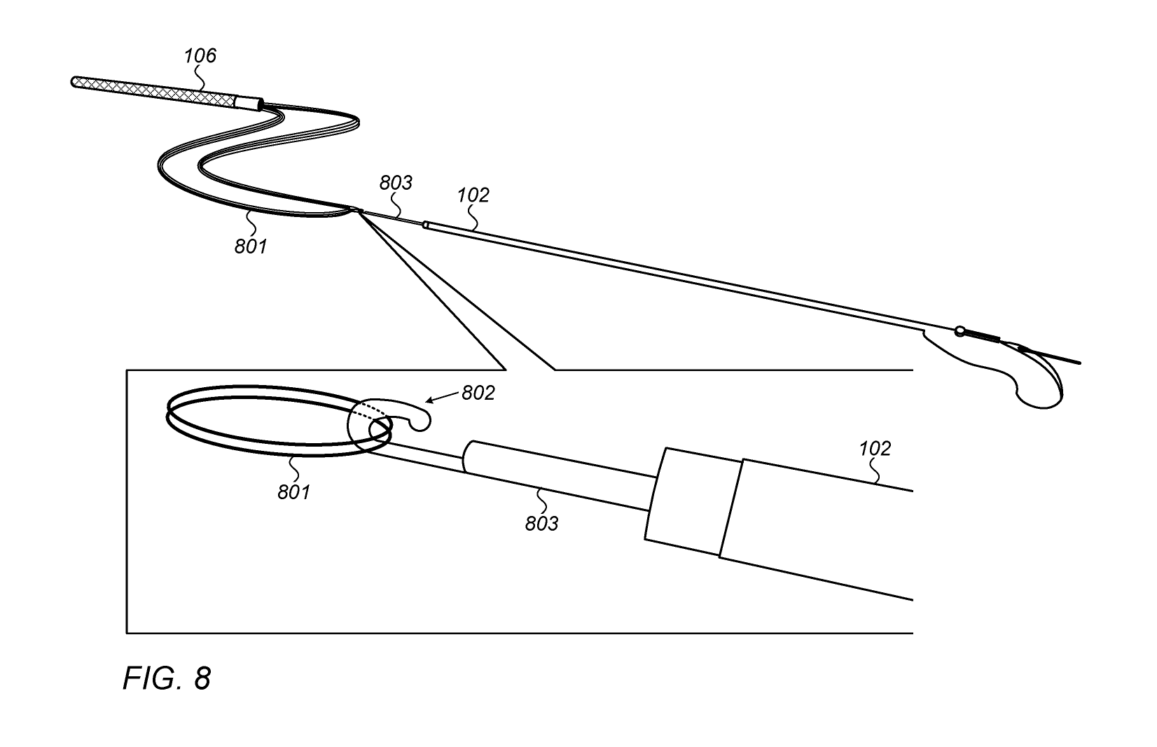

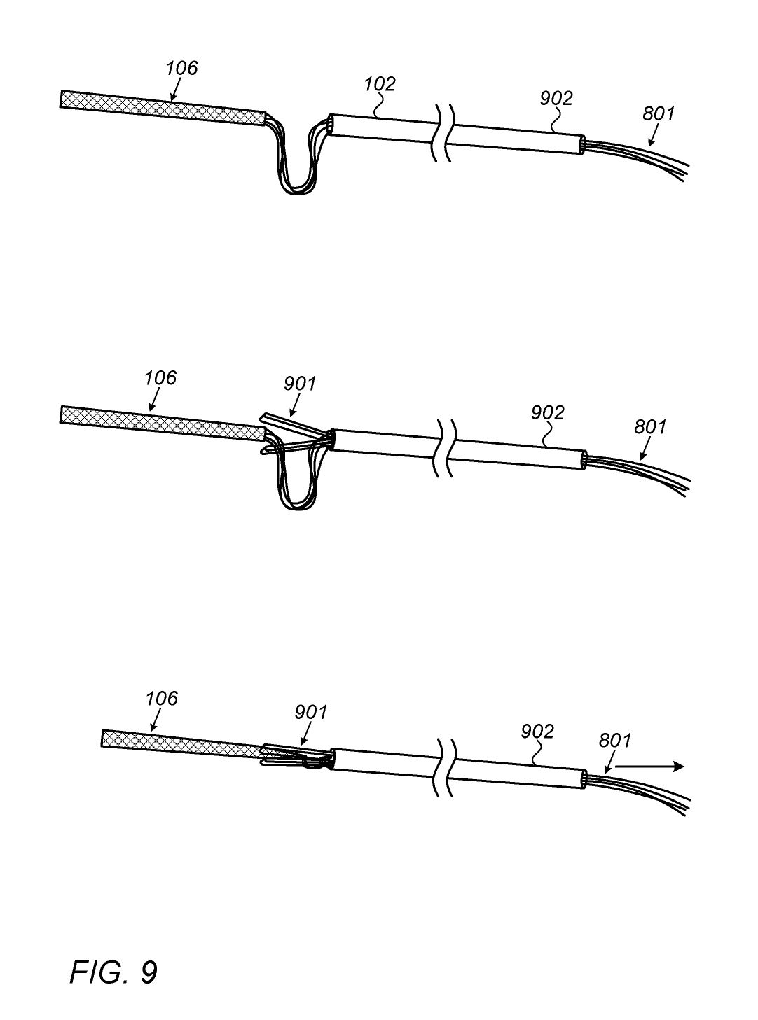

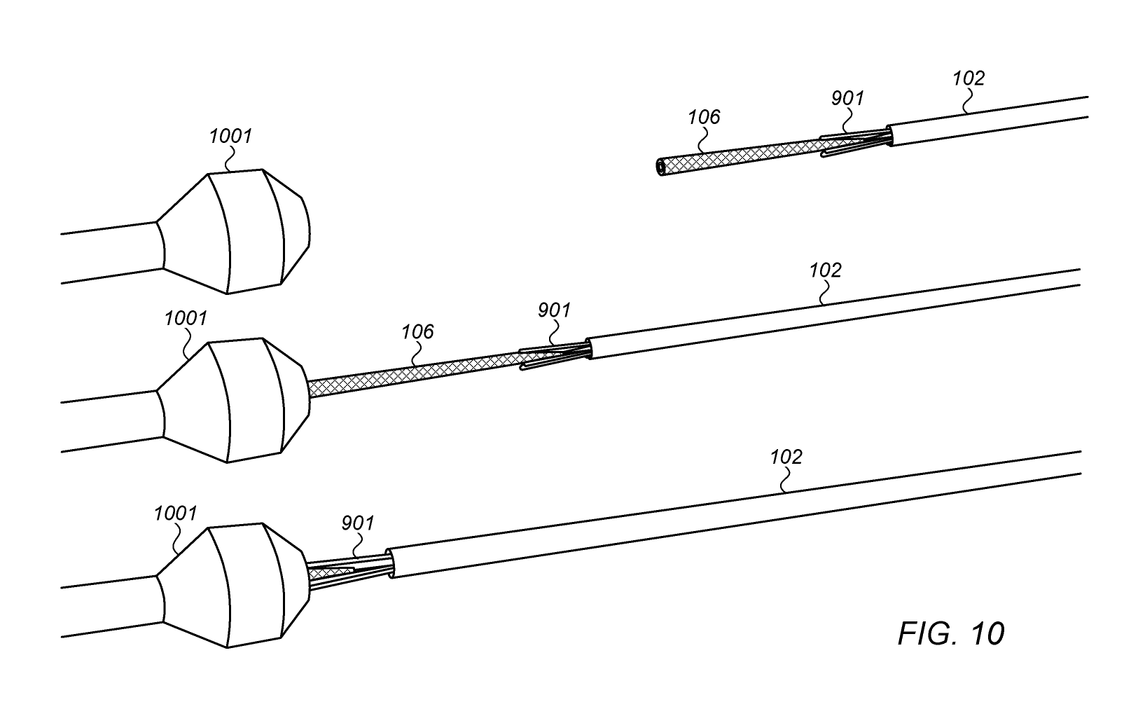

Optionally, staples, tacks, clips, hooks, and/or the like are placed on the mesh and/or tool prior to insertion into the surgical cavity. A surgical mesh may be prepared with suture threads and/or wires before insertion for use after insertion for deploying the mesh. FIG. 7 schematically shows surgical mesh 106 and wires 701 partially rolled or folded with the wires/sutures aligned with the rolling axis, such as extending from one side. FIG. 8 schematically shows a second tool 803 with a hook 802 used to pull wires 801 through a hollow center cavity of distal end 102 of the first tool. The rolled surgical mesh with the wires may be inserted into the first tool, with the wires exiting the proximal end of the tool. FIG. 9 schematically shows a clamp 901 at the distal end 102 of a surgical tool 902 before extension, after extension, and after clamping a surgical mesh 106 for insertion into a surgical cavity. Wires 801 may be pulled straight before inserting tool 902 into a surgical port. FIG. 10 schematically shows a surgical mesh 106 rolled and clamped onto a tool before, during and after insertion into a port 1001 leading from outside a subject body to a surgical cavity inside the subject body. Clamp 901 extends from distal end 102 of the tool.

Optionally, a placement and deployment are performed using wires, sutures, and/or the like, to attach a surgical mesh to a tool.

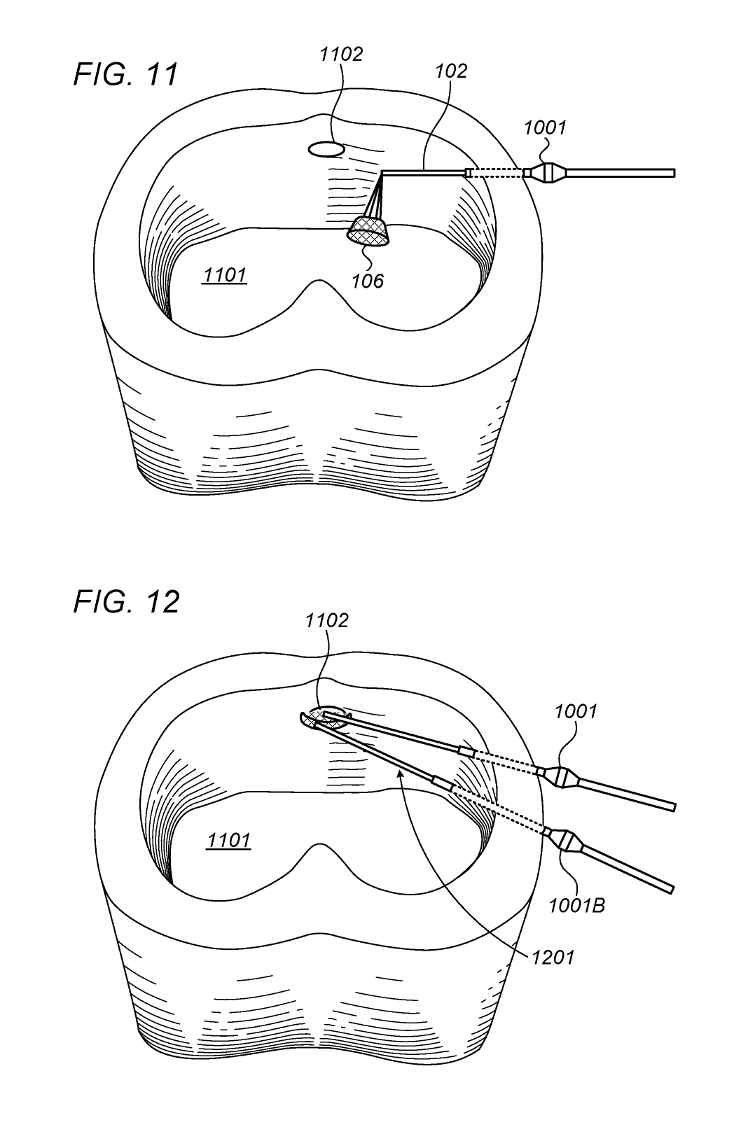

A rolled surgical mesh may be inserted into a port before insertion of a tool, and after the surgical mesh is inside the port the surgical tool may push the mesh through the port. FIG. 11 schematically shows a surgical mesh 106 partially deployed inside a surgical cavity 1101 and suspended in cavity 1101 from the wires of the deployment tool. The deployment may be achieved by positioning the distal end of the deployment at the hernia location 1102 and by pulling the wires in proximal direction using, e.g., a pulling mechanism, which results in lifting towards and placing of mesh 106 at hernia location 1102. FIG. 12 schematically shows a second tool 1201 used to align the surgical mesh with a hernia location 1102. Second tool 1201 may be inserted into a second port 1001B. A clamp tool and a cutting tool may be used to remove the wires and/or sutures from the surgical mesh. A rigid surgical tool may use one or more threads for deploying a surgical mesh in a surgical cavity.

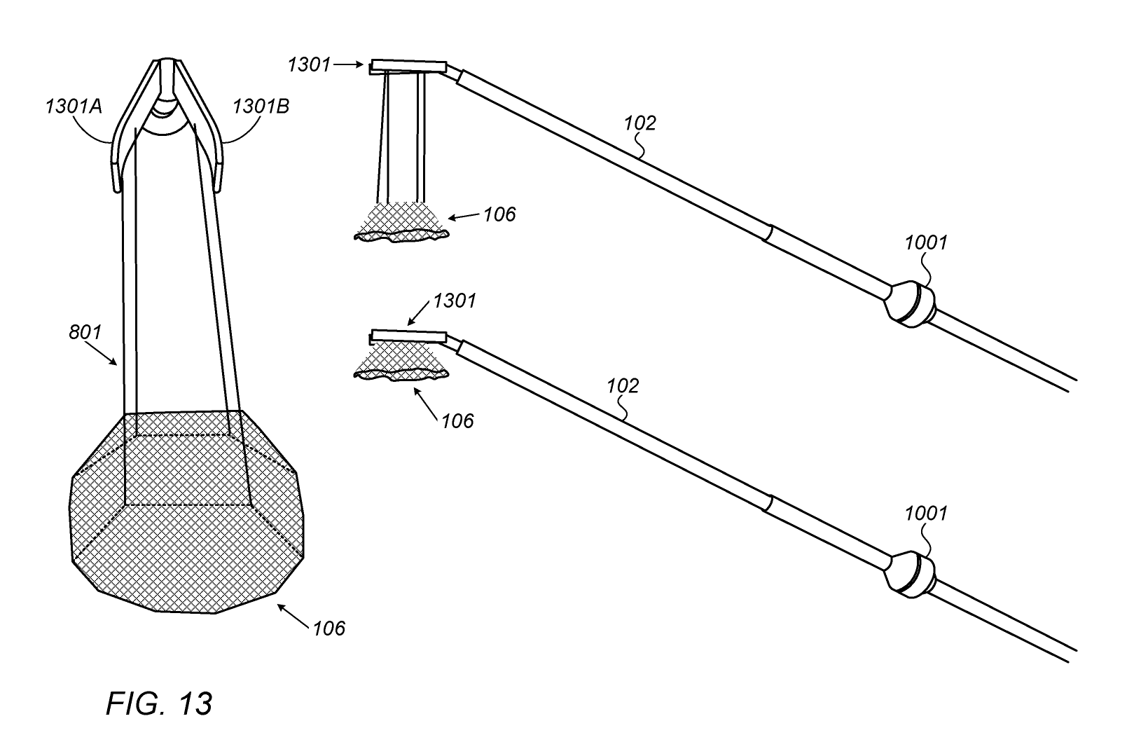

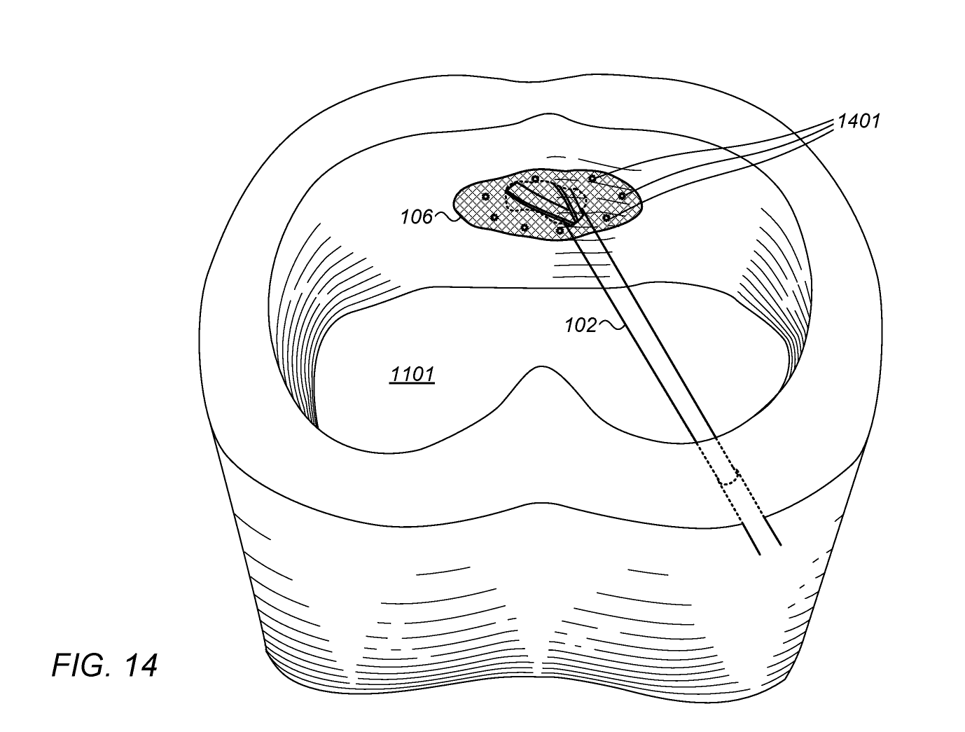

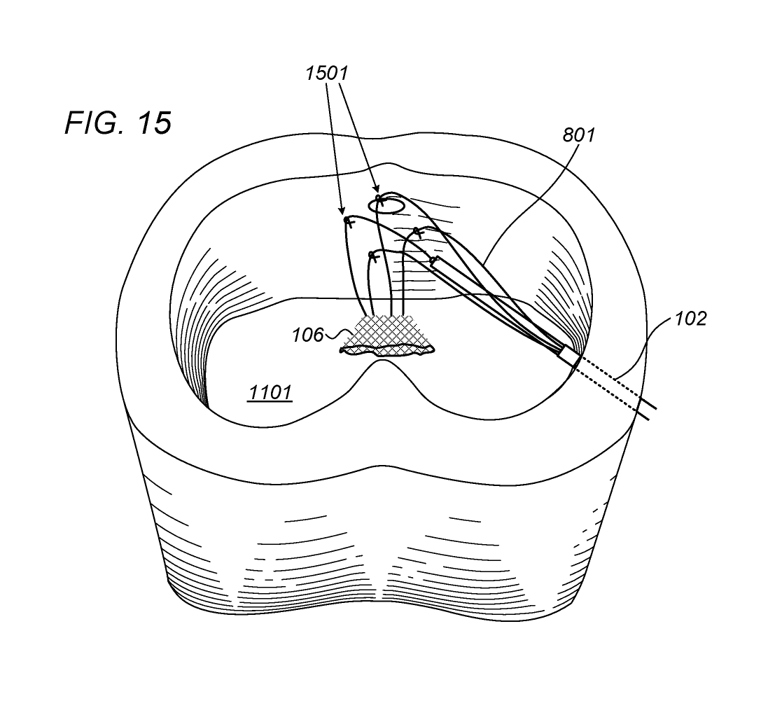

FIG. 13 schematically shows a surgical tool with two leg extensions 1301, as at 1301A and 1301B, for deploying a surgical mesh 106 using wires/sutures 801 from a distal end 102 of the tool, a side view of the tool with surgical mesh and long wires for transport within the surgical cavity, and a side view with surgical mesh and wires/sutures shortened for placement at the hernia location. Leg extensions 1301A and 1301B may be used to pulling surgical mesh 106 to the hernia location with wires/sutures 801 from two directions to better handle surgical mesh 106. FIG. 14 schematically shows surgical mesh 106 attached to the hernia location with tacks 1401. FIG. 15 schematically shows a deployed surgical mesh 106 being pulled by threads 801 to the hernia location with four anchors or hinges 1501. After surgical mesh 106 is positioned at the hernia location, a cutting tool may be used for removing the wires/sutures from the deployed surgical mesh.

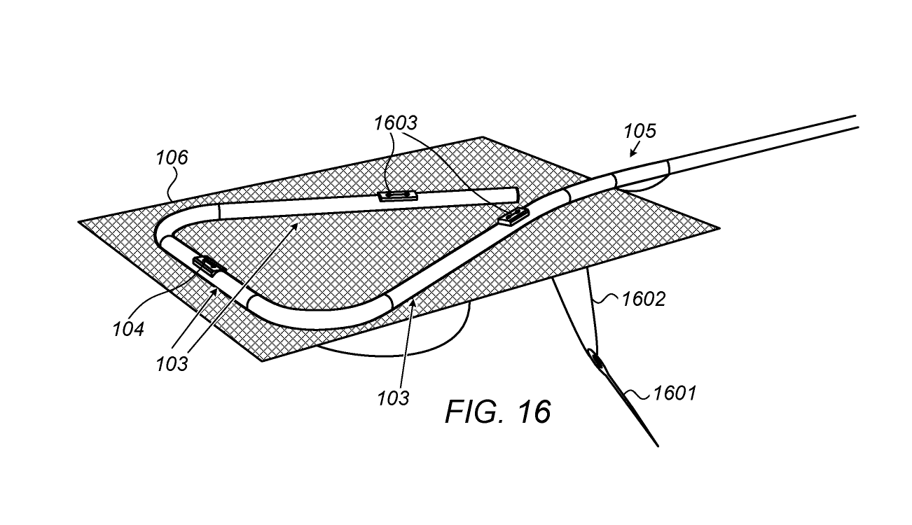

FIG. 16 schematically shows a surgical mesh 106 being attached to a surgical tool 105 using thread, sutures, and/or wires 1603, optionally, using a needle 1601 and thread 1602 for suturing the mesh to wires 1603. The mesh may be attached to a surgical tool using wires, and placed in the surgical cavity via an opening, such as a port, while remaining coupled using the wires to the distal part of the surgical tool. The wires may be pulled from outside the cavity using the proximal part of the tool. The distal tool part may be inserted in its collapsed configuration into the surgical cavity so that both the distal tool part and the mesh are in the cavity. The distal tool part located in the surgical cavity may then be converted to the frame structure in the expanded configuration. When in the frame structure, the mesh may be attached at various points to the frame. These attachment points may be at positions that correspond to the form of the frame, i.e., match the frame geometry.

The wire may be pulled to release the surgical mesh and attached the surgical mesh after being attached to a hernia location using pins. Optionally, by partially pulling the wires, the mesh is pulled against and attached to the frame in an expanded configuration. When attached to the frame, the mesh may be placed at the hernia location and secured, such as using tacks, and the wire pulled fully to release the mesh.

When the mesh is coupled to a tool, the mesh may be placed onto the herniated tissue in various ways. In one example, the mesh may be placed onto the herniated tissue such that the tool is between the mesh and the hernia. Once the mesh is secured onto the tissue, the tool may be retracted, i.e., pulled out from the position adjacent to the hernia. In another example, the mesh may be positioned between the tool and the tissue with the hernia. Once the mesh is secured to herniated tissue, the tool is decoupled from the secured mesh.

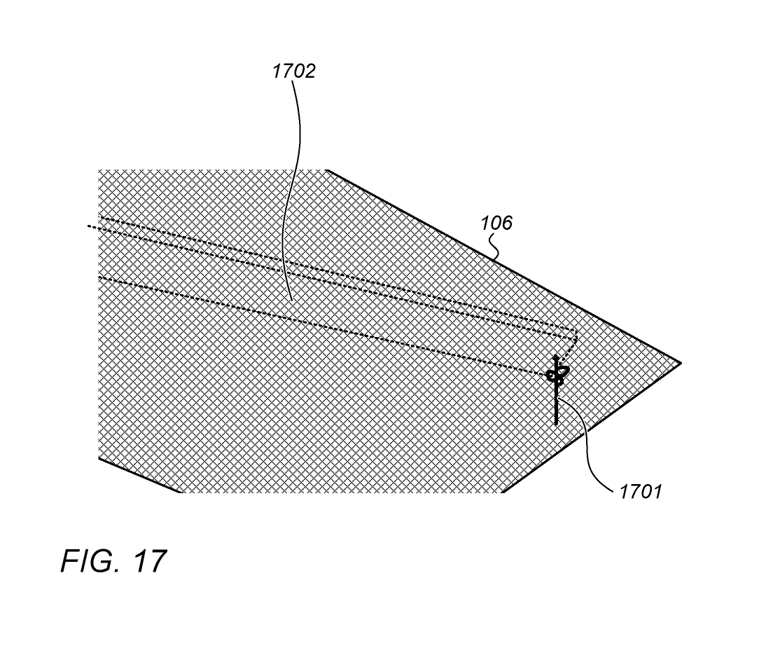

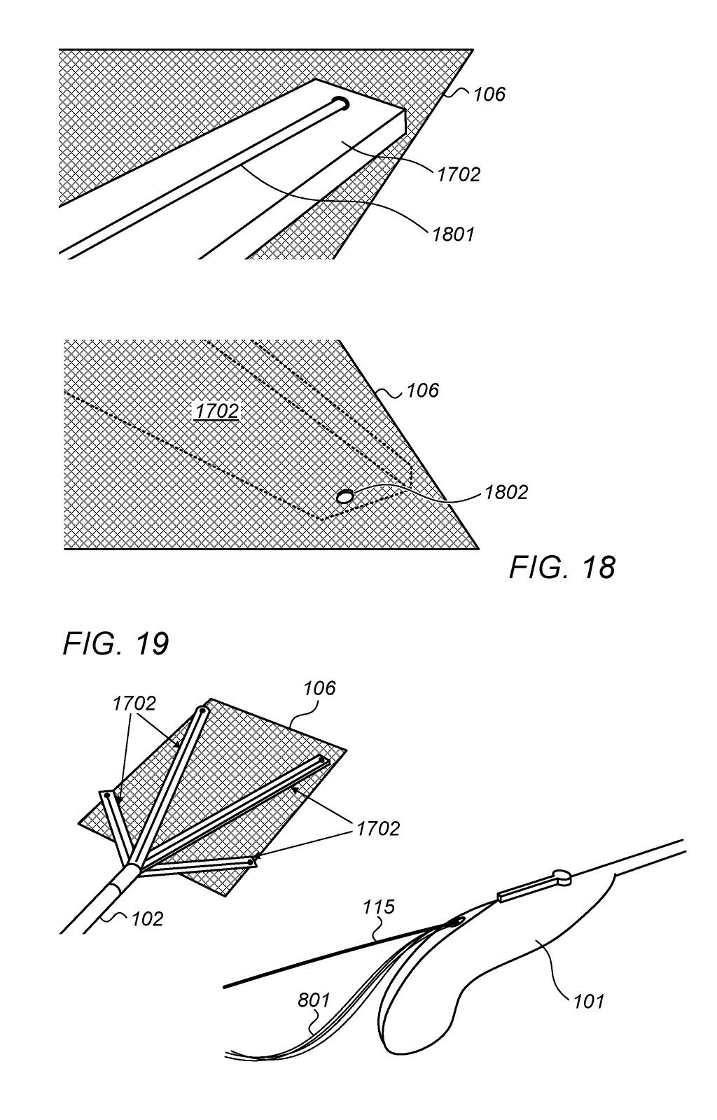

FIG. 17 schematically shows a front side of one leg 1702 of a multiple leg surgical tool with a surgical mesh 106 attached using sutures 1702. FIG. 18 schematically shows a front and back side of one leg 1702 of a multiple leg surgical tool with a surgical mesh 106 attached using a wire 1801 with ball ends 1802.

FIG. 19 schematically shows proximal 101 and distal ends 102 of a multiple leg 1702 surgical tool with a surgical mesh 106 attached using a wire 115 and sutures 801. Sutures 801 may hold mesh 106 to the surgical tool and wire 115 may release the mesh from the tool when pulled. Additional or alternative attachment mechanisms for attaching the mesh to the frame may be employed, as outlined herein.

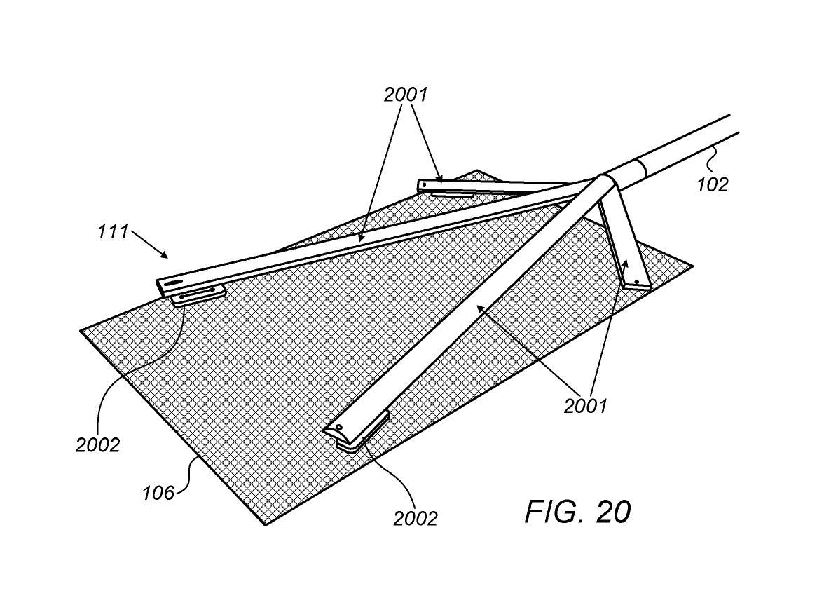

Optionally, a placement and deployment are performed using magnets to attach a surgical mesh to a tool. FIG. 20 schematically shows a surgical mesh 106 being attached to a multiple leg 2001 surgical tool with magnets 2002. Magnets 2002 may comprise a proximal magnet pair and a distal magnet pair. The proximal magnet pair may have a different angular orientation with respect to a longitudinal extension of a distal end 102 of the tool than the distal magnet pairs to match the orientation of the legs.

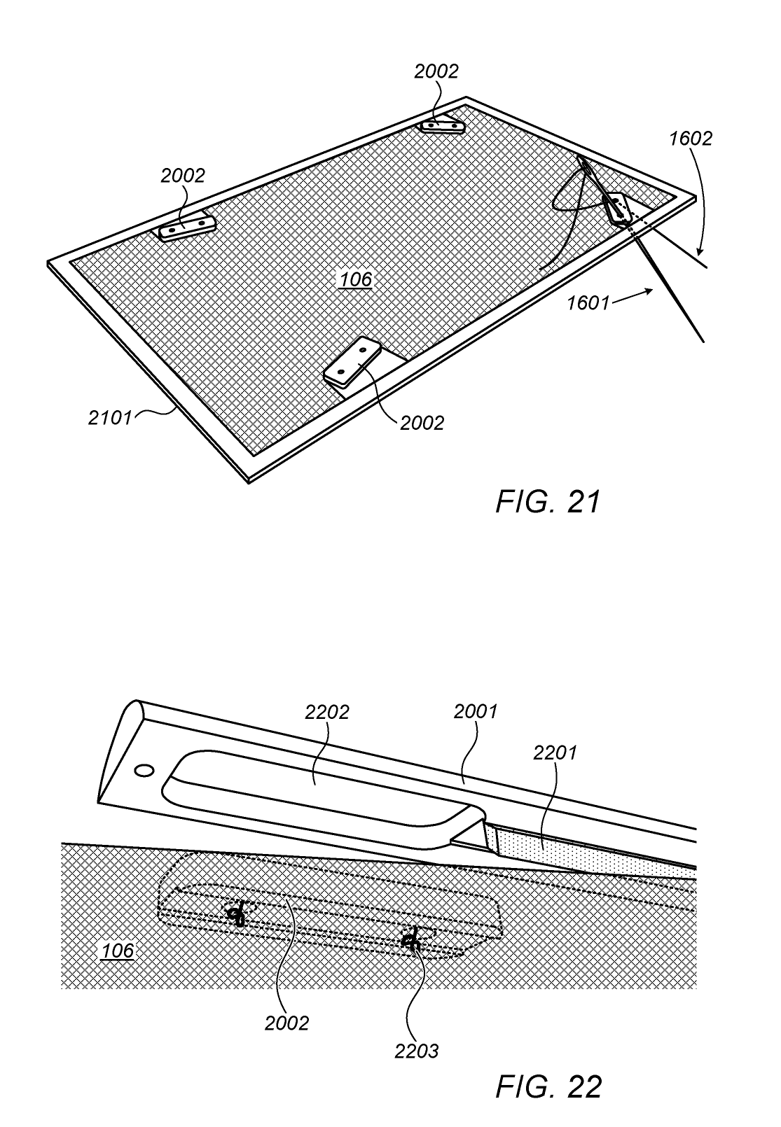

Optionally, a jig is used to attach magnets to a surgical mesh, such as with a needle and thread. FIG. 21 schematically shows magnets 2002 that are attached to a jig 2101 with a mesh 106 in an expanded configuration for enabling the jig 2101 to position magnets 2002 while being sutured using needle 1601 and thread 1602. FIG. 22 schematically shows a close-up view of a leg 2001 of a surgical tool for clamping surgical mesh 106 with magnets 2002 to a recess 2202 in leg 2001 configured to receive magnet 2002. Recess 2202 may be configured to receive the shape of magnet 2002, have ferromagnetic elements to attract magnet 2002, have magnetic elements to attract magnet 2002, and the like. For example, the north and south poles of magnet 2002 may match the north and south poles of a magnetic element in recess 2202. The surgical mesh may be secured to the magnets by suture threads 2203, and leg 2001 may include a blade 2201 to cut the threads and release mesh 106. Magnet 2002 may be retained in recess 2202 and extracted through the surgical port. FIG. 23 schematically shows a cutting blade 2201 insert in the leg 2001 of a surgical tool before during and after cutting the suture wires 2301 attaching a magnet 2002 to the surgical mesh 106. The fourth view shows surgical mesh 106 detached from the tool.

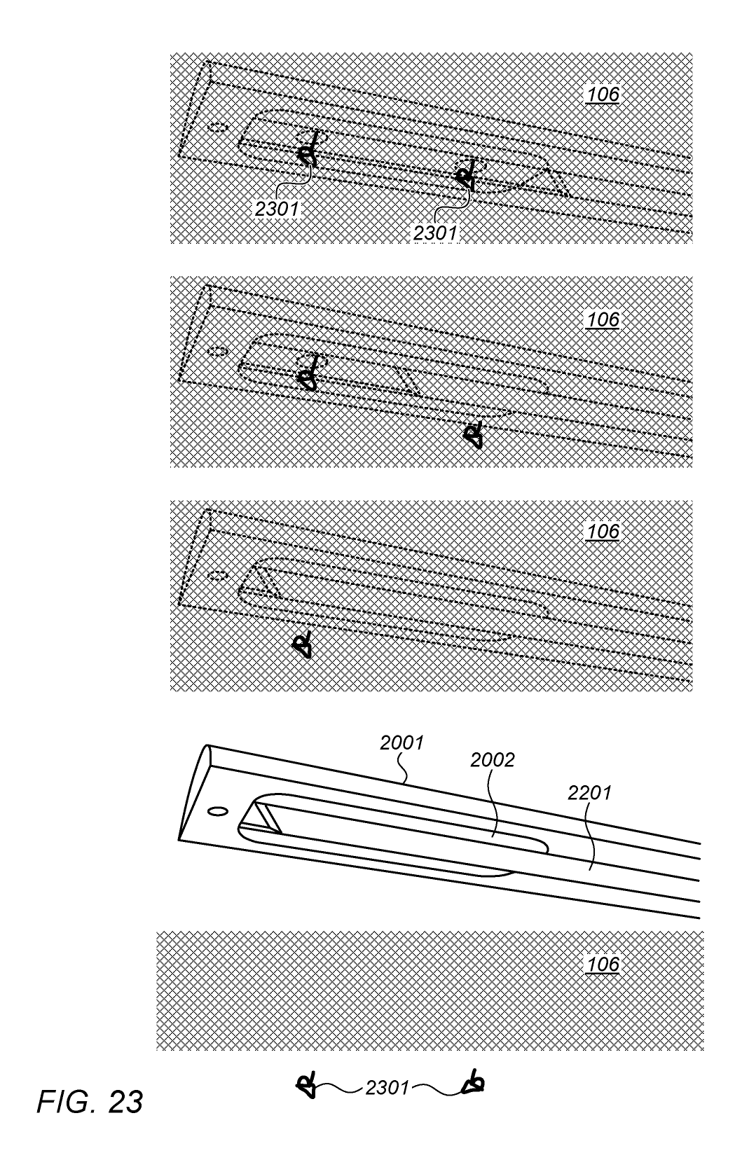

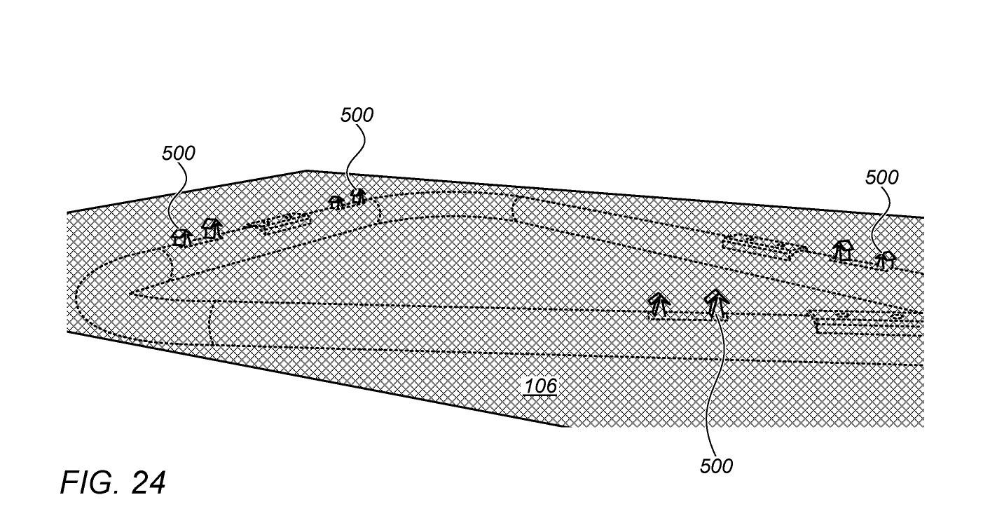

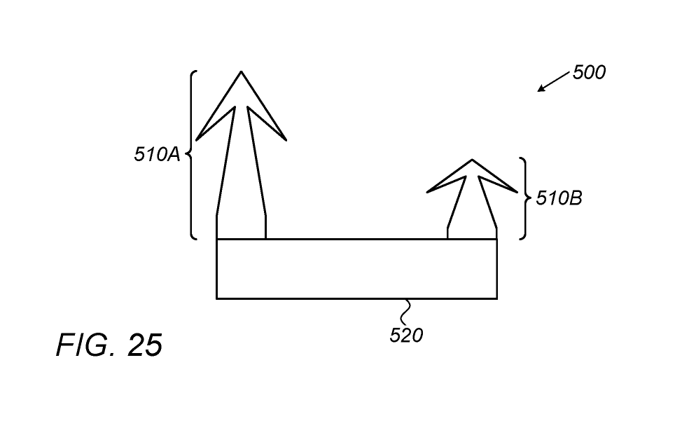

FIG. 24 schematically shows a surgical tool with surgical mesh 106 attached and anchors/tacks 500 ready for positioning and attaching to a tissue surrounding a hernia using anchors/tacks 500. FIG. 25 is a schematic enlarged view of an example coupling element embodied by an anchor/tack 500 which comprises two barbed legs 510A and 510B extending from base 520. Each barbed leg is in substantially the same direction and they are substantially parallel to each other. First anchor leg 510A may be longer than second anchor leg 510B. Both first and second anchor legs 510A and 510B may each have a flexible barb to disengage from tissue by retraction. The different lengths of anchor legs 510A and 510B may facilitate removal of anchor 500 by pulling out and redeploy them into the tissue when required, with less force compared to the pulling force that may be required if both anchors were of the longer length.

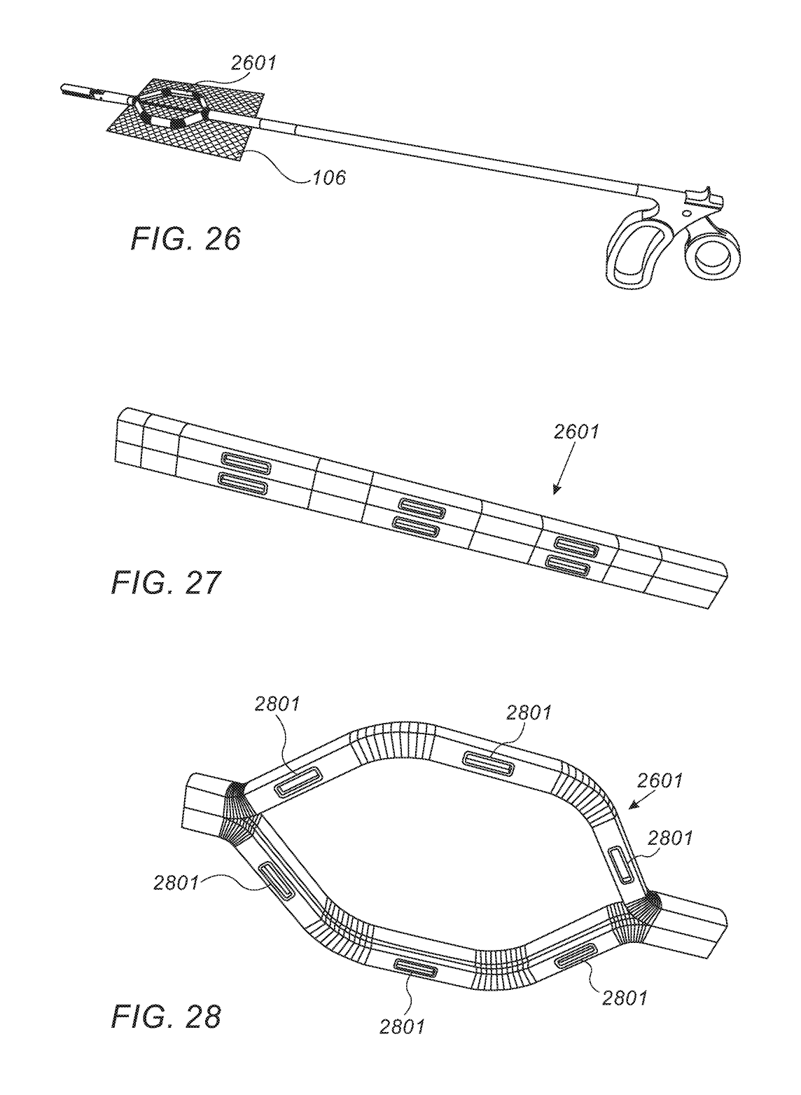

Optionally, a placement and deployment are performed using suction and/or vacuum to attach a surgical mesh to a tool. FIG. 26 schematically shows a surgical tool with the surgical mesh 106 attached by suction through an expandable suction frame 2601. A recess in suction frame 2601 may be used for positioning the surgical mesh within the surgical cavity by coupling mesh 106 to frame 2601 using suction through a hollow portion of frame 2601 and tool. After insertion into the surgical cavity, surgical mesh 106 may be spread and coupled, e.g. by vacuum and/or magnets, to frame 2601.

FIG. 27 schematically shows a collapsed configuration of a surgical tool for attaching the surgical mesh by suction. Optionally, the expanded configuration comprises a closed loop shape at the distal end of the surgical tool. Optionally, the expanded configuration comprises a closed loop shape and central straight section at the distal end of the surgical tool. FIG. 28 schematically shows an expanded configuration of a closed loop frame 2601 for attaching the surgical mesh by suction. Suction is applied to the mesh through outlets 2801 along frame 2601.

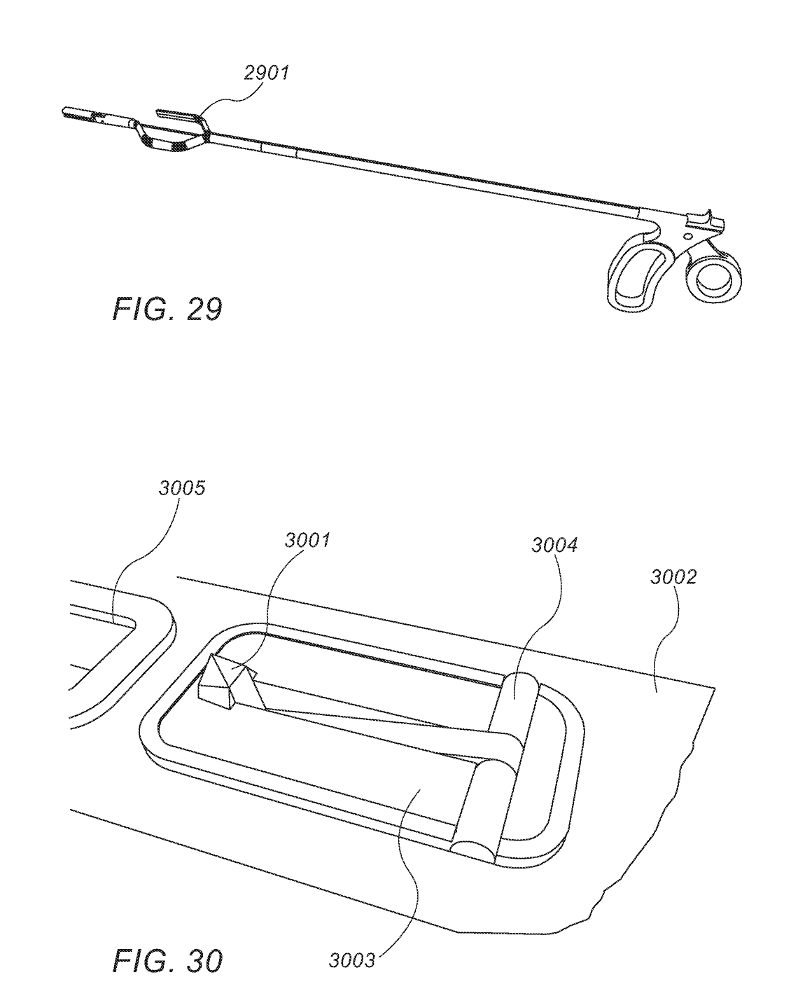

Optionally, the expanded configuration comprises an open loop shape frame. FIG. 29 schematically shows an expanded configuration of an open loop frame 2901 surgical tool for attaching the surgical mesh by suction.

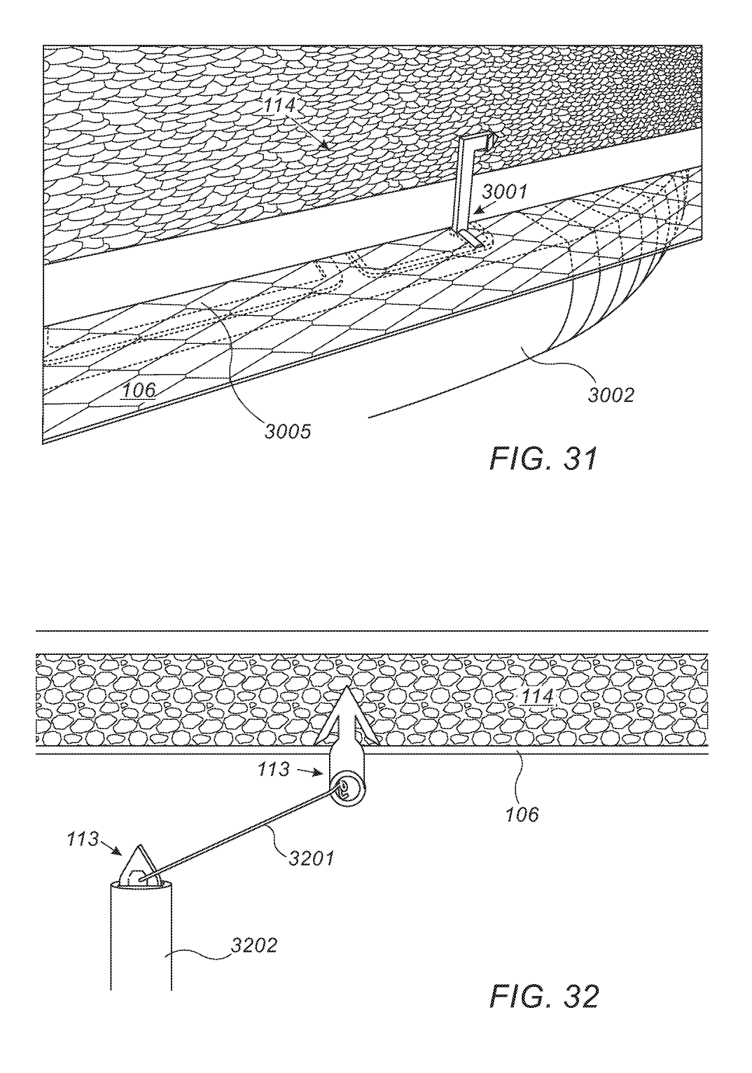

Optionally, the surgical tool has recesses containing detachable coupling elements (e.g., hooks/anchors/tacks) for securing a mesh to a tissue. FIG. 30 schematically shows hooks 3001 in retracted position, such as anchors flush to and embedded in frame 3002. Hook 3001 may rotate around a pivot 3004, both of which are collapsed in a recess 3003 adjacent to a suction port 3005. FIG. 31 schematically shows a hook 3001 in anchored position in which the hooks/anchors protrude from the frame 3002 allowing driving anchors 3001 into the tissue 114 surrounding a hernia “from below”.

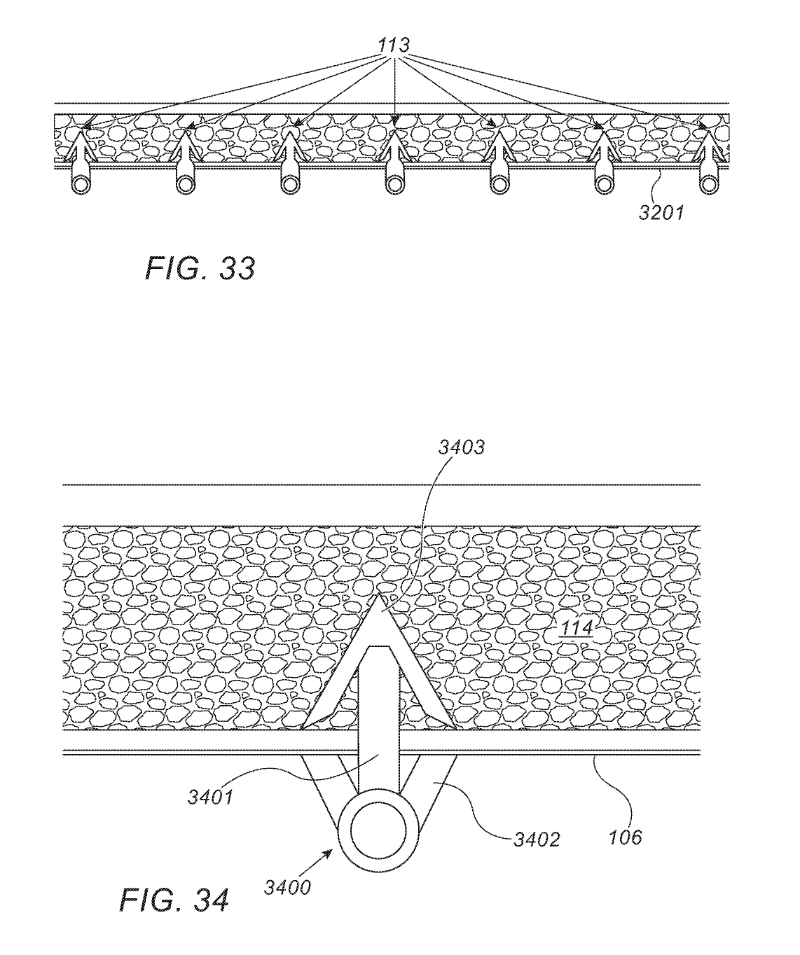

Optionally, the hook anchors are deployed with a dedicated tool, such as a hook deployer. FIG. 32 schematically shows a hook deploying tool 3202 and a hook 113 securing a mesh 106 to a tissue 114. Hooks 113 may be connected by a thread 3201 and/or wire for anchoring or removal. FIG. 33 schematically show rows of hooks 113 connected with a thread 3201 for securing a mesh to a tissue.

Optionally, the anchors are textured, hook-shaped, barb-shaped, and/or the like. For example, the hooks have a barbs to prevent the anchors from being pulled back from the tissue region by retraction, such as a harpoon-like point. Optionally, pairs of neighboring hooks are coupled with each other via a wire, suture, thread, and/or the like.

FIG. 34 schematically shows a hook 3400 with a barbed point 3403 for entering a tissue 114, a shaft 3401, and a mesh anchor 3402 for securing a surgical mesh 106 to tissue 114.

Optionally, coupling elements for securing a mesh to a tissue have a U-shaped configuration comprising two staple legs with leg ends and a crossbar joining the two legs. The coupling elements may be deployed using the dedicated tool by driving the staple legs into the tissue. The legs driven into the tissue may bend, e.g., towards each other, into a folded configuration to secure the mesh to the tissue through clinching. Optionally, the U-shaped coupling element may be made of shape-memory material (SMM) such as, for example, Nitinol. For example, the SMM-based staple may be cooled and then deformed into the U-shape body while at a first temperature which is less than the transformation temperature at which it is in the martensitic phase. The U-shaped staple is then inserted in its deformed shape and attains a comparably elevated temperature to reform to its original shape. The elevated temperature may be obtained through heating, e.g., by a heating element (not shown) which may for example be comprised in the delivery system, or responsive to attaining the temperature of the surrounding tissue. In another example, the staple may be deformed and inserted into the tissue while being held in the deformed state at a temperature such that it automatically attempts to reform to its original shape.

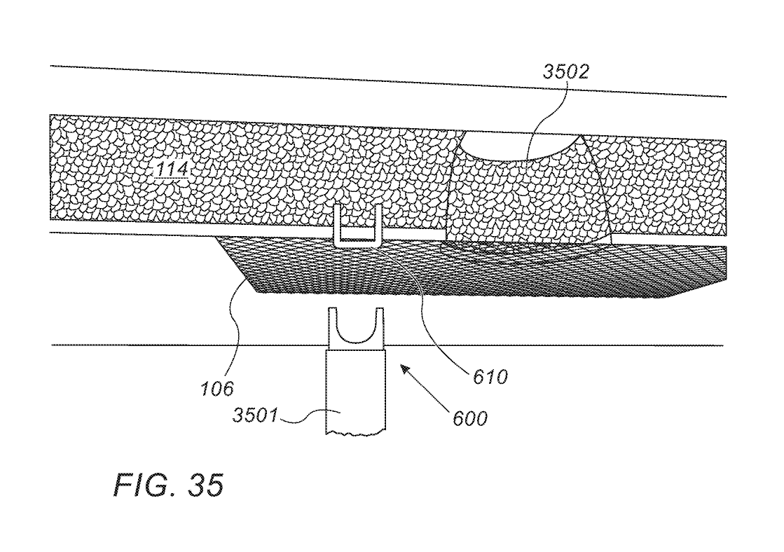

FIG. 35 schematically shows a mesh coupling element delivery tool 600 for deployment of a U-shaped coupling element 610 through a distal end 3501 of tool 600. Coupling elements 610 are deployed to secure a surgical mesh 106 to tissue 114 near a hernia 3502 through clinching of tissue and mesh by the legs and crossbar of the elements.

In some embodiments, the surgical tool be operative to receive the dedicated tool 600, for deploying coupling elements 601 and securing mesh 106 to tissue 114. For example, the surgical tool may have a tube shaped body 3501 enclosing a cavity longitudinally extending within the surgical tool when the tool is in the collapsed configuration. When the surgical tool is in the expanded configuration, the dedicated tool 600 may traverse across a plane delineated and enclosed by a frame portion that may be formed by a portion of the surgical instrument for holding the mesh. A distal end of dedicated tool 600 may protrude from the distal end 102 of surgical instrument 100 and may be manipulated by a handle of surgical instrument 100, e.g., for the deployment of coupling elements.

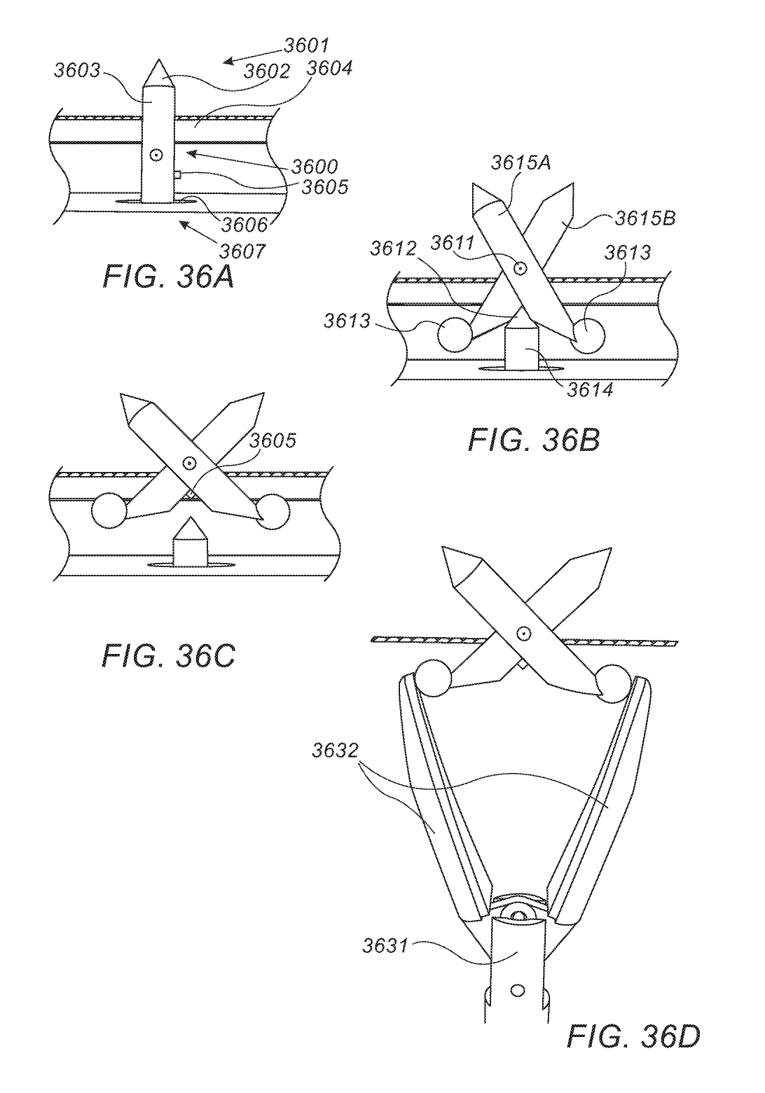

FIGS. 36A-36D schematically shows a removable cross-shaped tack in different configurations. In FIGS. 36A a removable tack 3600 is in a closed configuration for insertion though a surgical mesh 3604 into a tissue 3601. The distal end 3602 of removable tack 3600 is pointed to easily penetrate mesh 3604 and tissue 3601. A locking tooth 3605 is located along the shaft 3603 of tack 3600. Tack 3600 is released from a tool 3607 through a port 3606.

FIG. 36B schematically shows tack 3600 in a partially open configuration. When tack 3600 is fully inserted into tissue, a protrusion 3614 exits port 3606, and forces two halves 3615A and 3615B of tack 3600 to pivot relative to each other about a pivot pin 3611. Distal end 3612 of protrusion 3614 is wedge shaped to force two halves 3615A and 3615B apart. Blunt proximal ends 3613 of tack 3600 assist in securing an object, such as surgical mesh 3604 to tissue 3601.

FIG. 36C schematically shows tack 3600 in an open configuration, after protrusion 3614 has fully expanded tack 3600 with locking tooth 3605 preventing the pivoted halves 3615A and 3615B from closing. FIG. 36D schematically shows a tool 3631 removing tack 3600 by compressing blunt ends 3613 with clamps 3632.

After being temporarily affixed, the mesh may be secured, e.g., using the staples described herein by employing a dedicated tool which is delivered into the body lumen via the instrument cavity of the same surgical instrument which is used for deploying the mesh.

Following is a description of a tool for blocking an artery.

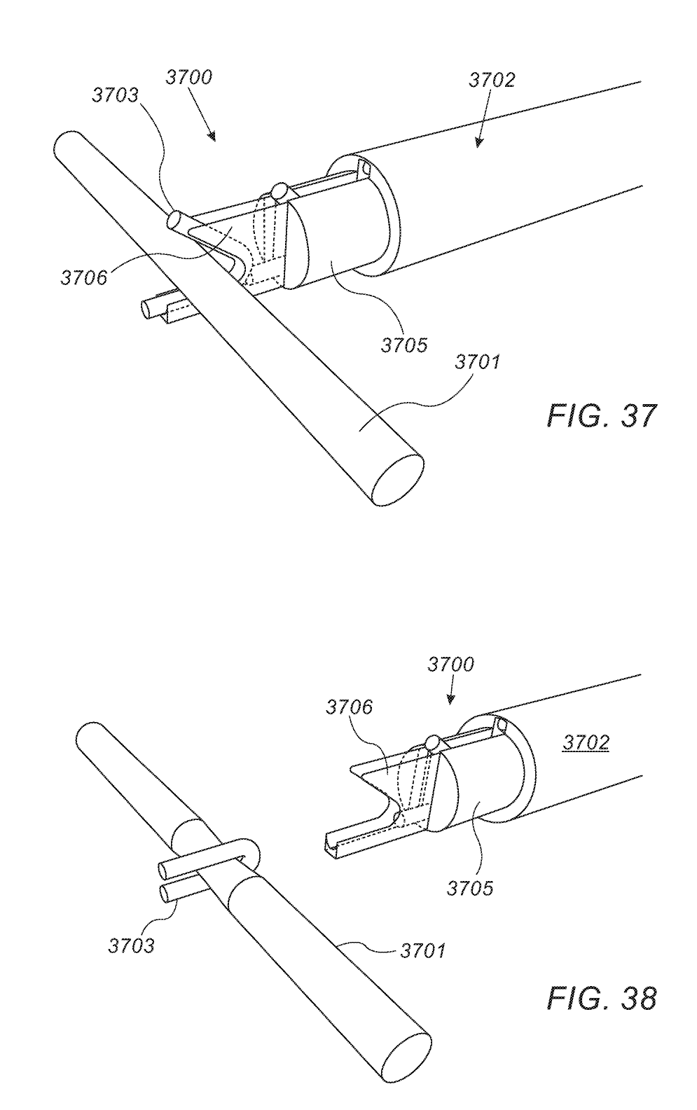

Reference is made to FIGS. 36 and 37. FIG. 36 schematically shows a tool 3700 deploying a blocking element 3703 in expanded configuration. FIG. 37 shows blocking element 3703 in collapsed configuration, clamping a blood vessel 3701 and a tool 3700 retracting. In an embodiment, a vessel blocking element 3703 for blocking blood vessel 3701 may have, in an open position, a V-shaped configuration having two legs of which two common leg ends are coupled to each other. Vessel blocking element 3703 may be set in a closed position in which the two legs proximate to clamp onto a blood vessel to be blocked. Blocking element 3703 may thus, in an embodiment, act like a vise. Blocking element 3703 may be made of shape memory material in which the closed position may be referred to as the “original shape”. Vessel blocking element 3703 may attain its closed, original, shape when the material's temperature has risen above a threshold required for the element to assume its original shape. Element 3703 may exit a distal end 3702 of tool 3700, and be collapsed on vessel 3701 using a manipulator 3706.

In an embodiment, tool 3700 may comprise a heating element 3705 for heating up vessel blocking element 3703 such that element 3703 is re-formed from a deformed (expanded configuration) to its original shape.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A system comprising:
an elongated mesh deployment tool having a proximal tool part and a distal tool part; and
at least one wire for coupling a surgical mesh with the distal tool part, wherein the wires are coupled to the surgical mesh and the distal tool part in at least one location;
wherein the distal tool part is configured for deploying the surgical mesh at a hernia location, wherein the deploying is performed by pulling the at least one wire in a proximal direction to mount the surgical mesh in a spread configuration near the hernia location.

2. The system of claim 1, wherein the at least one wire extends from outside a surgical cavity into the surgical cavity and are configured to spread the surgical mesh which is located in the surgical cavity while the distal tool part is outside the surgical cavity.

3. The system of claim 1, wherein in an expanded configuration the distal tool part has a polygon-shaped frame-like structure.

4. The system of claim 3, wherein the distal tool part comprises sections that are bendable relative to each other to form the polygon-shaped frame-like structure.

5. The system of claim 1, wherein the distal tool part comprises a plurality of legs, wherein in the expanded configuration at least two of the plurality of legs are oriented for spreading the surgical mesh.

6. The system of claim 5, wherein the plurality of legs comprises three or more legs that terminate in the expanded configuration in end points that lie substantially co-planar for spreading the surgical mesh.

7. The system of claim 1, wherein the surgical mesh is attached to the expanded distal tool part using any one of sutures, magnets, and/or suction.

8. The system of claim 1, further comprising anchors deployable by the distal tool part at the boundaries of a herniated tissue, wherein in a surgical cavity the at least one wire runs from the surgical mesh to the anchors, and from the anchors to a distal tool section so that by pulling the at least one wire, the surgical mesh suspends from the anchors and is lifted towards the anchors for attachment at the hernia location.

9. The system of claim 1, further comprising a pulling mechanism located at a proximal end of the mesh deployment tool, wherein the pulling mechanism is configured for pulling the at least one wire from outside the surgical cavity in a proximal direction.

10. The system of claim 1, further comprising a tacking mechanism located at the distal tool part configured for inserting tacks through the mesh into the tissue surrounding the hernia location.

11. The system of claim 10, wherein the tacks are configured to be biodegradable.

12. The system of claim 10, wherein the tacks are configured to be removed easily for repositioning the mesh.

13. The system of claim 10, wherein the tacks comprise a plurality of barbs, each barb of a different length.

14. A system comprising:
an elongated mesh deployment tool having a proximal tool part and a distal tool part, wherein the distal tool part comprises a mesh clamp; and
at least one wire for coupling a surgical mesh with the distal tool part, wherein the at least one wire is coupled to the surgical mesh and the distal tool part in at least one location;
wherein the mesh clamp comprises (i) a collapsed configuration configured for minimally invasively insertion through a port into a surgical cavity, wherein the mesh clamp in the collapsed configuration grips a surgical mesh, and (ii) an expanded configuration configured for attaching the surgical mesh to the distal tool part, wherein the attaching is performed by pulling the at least one wire in a proximal direction to mount the surgical mesh in a spread configuration onto the distal tool part, and the expanded configuration is further configured for deploying the surgical mesh at a hernia location.

15. The system of claim 14, wherein the at least one wire extends from outside a surgical cavity into the surgical cavity and are configured to spread the surgical mesh which is located in the surgical cavity while the distal tool part is outside the surgical cavity.

16. The system of claim 14, further comprising anchors deployable by the distal tool part at the boundaries of a herniated tissue, wherein in a surgical cavity the at least one wire runs from the surgical mesh to the anchors, and from the anchors to the distal tool section so that by pulling the at least one wire, the mesh suspends from the anchors and is lifted towards the anchors for attachment at the hernia location.

17. The system of claim 14, further comprising a pulling mechanism located at a proximal end of the mesh deployment tool, wherein the pulling mechanism is configured for pulling the at least one wire from outside the surgical cavity in a proximal direction.

18. The system of claim 14, further comprising a tacking mechanism located at the distal tool part configured for inserting tacks through the mesh into the tissue surrounding the hernia location.

19. The system of claim 18, wherein the tacks are configured to be biodegradable.

20. The system of claim 18, wherein the tacks are configured to be removed easily for repositioning the mesh.

21. The system of claim 18, wherein the tacks comprise a plurality of barbs, each barb of a different length.

22. A system comprising:
an elongated mesh deployment tool having a proximal tool part and a distal tool part; and
a plurality of legs, each leg extending from the distal tool part and configured to be strongly attracted to a magnet;
wherein each leg comprises a slicing blade embedded therein, and each leg couples magnetically to a surgical mesh comprising a plurality of corresponding magnets secured to the surgical mesh with thread; and
wherein the plurality of legs comprises (i) a collapsed configuration configured for minimally invasively insertion through a port into a surgical cavity, and (ii) an expanded configuration configured for attaching the surgical mesh to the distal tool part, wherein the attaching is performed by mount the surgical mesh in a spread configuration onto the plurality of legs using magnetic attraction, and the expanded configuration is further configured for deploying the surgical mesh at a hernia location, and wherein when the surgical mesh is located at the hernia location the slicing blades slice each of the threads securing magnets to the surgical mesh.

23. The system of claim 22, wherein in the expanded configuration the distal tool part has a polygon-shaped frame-like structure.

24. The system of claim 23, wherein the distal tool part comprises sections that are bendable relative to each other to form the polygon-shaped frame-like structure.

25. The system of claim 22, further comprising a tacking mechanism located at the distal tool part configured for inserting tacks through the mesh into the tissue surrounding the hernia location.

26. The system of claim 25, wherein the tacks are configured to be biodegradable.

27. The system of claim 25, wherein the tacks are configured to be removed easily for repositioning the mesh.

28. The system of claim 25, wherein the tacks comprise a plurality of barbs, each barb of a different length.

29. A system comprising:
an elongated mesh deployment tool having a proximal tool part and a distal tool part, wherein the distal tool part comprises a plurality of bending sections and a plurality of suction orifices;
wherein each suction orifice is configured to couple with part of a surgical mesh by applying suction to the proximal tool part; and
wherein the distal tool part comprises (i) a collapsed configuration configured for minimally invasively insertion through a port into a surgical cavity, and (ii) an expanded configuration configured for attaching the surgical mesh in a spread configuration to the distal tool part by applying the suction, and the expanded configuration is further configured for deploying the surgical mesh at a hernia location.

30. The system of claim 29, wherein in the expanded configuration the distal tool part has a polygon-shaped frame-like structure.

31. The system of claim 30, wherein the distal tool part comprises sections that are bendable relative to each other to form the polygon-shaped frame-like structure.

32. The system of claim 29, further comprising a tacking mechanism located at the distal tool part configured for inserting tacks through the mesh into the tissue surrounding the hernia location.

33. The system of claim 32, wherein the tacks are configured to be biodegradable.

34. The system of claim 32, wherein the tacks are configured to be removed easily for repositioning the mesh.

35. The system of claim 32, wherein the tacks comprise a plurality of barbs, each barb of a different length.

36. A system comprising:
an elongated mesh deployment tool having a proximal tool part and a distal tool part, wherein the distal tool part comprises a plurality of bending sections;
wherein each bending section is configured for attaching a surgical mesh using thread; and
wherein the distal tool part comprises (i) a collapsed configuration configured for minimally invasively insertion through a port into a surgical cavity with the surgical mesh attached, and (ii) an expanded configuration configured for deploying the surgical mesh at a hernia location.

37. The system of claim 36, wherein in the expanded configuration the distal tool part has a polygon-shaped frame-like structure.

38. The system of claim 37, wherein the distal tool part comprises sections that are bendable relative to each other to form the polygon-shaped frame-like structure.

39. The system of claim 36, further comprising a tacking mechanism located at the distal tool part configured for inserting tacks through the mesh into the tissue surrounding the hernia location.

40. The system of claim 39, wherein the tacks are configured to be biodegradable.

41. The system of claim 39, wherein the tacks are configured to be removed easily for repositioning the mesh.

42. The system of claim 39, wherein the tacks comprise a plurality of barbs, each barb of a different length.

43. A system comprising a mesh deployment tool, wherein the mesh deployment tool comprises a proximal tool part and a distal tool part, wherein the distal tool part is configured to be inserted into a surgical cavity through a port, and wherein the distal tool part is configured to be expanded for attaching a surgical mesh to the distal tool part in a spread configuration allowing deployment of the surgical mesh at a hernia location.

44. The system of claim 43, further comprising a tacking mechanism located at the distal tool part configured for inserting tacks through the mesh into the tissue surrounding the hernia location.

45. The system of claim 44, wherein the tacks are configured to be biodegradable.

46. The system of claim 44, wherein the tacks are configured to be removed easily for repositioning the mesh.

47. The system of claim 44, wherein the tacks comprise a plurality of barbs, each barb of a different length.
